(19) United States
(12) Patent Application Publication
(10) Pub. No.: US 2018/0311274 A1
(43) Pub. Date: Nov. 1, 2018

(54) IN SITU NEUTRALIZATION COMPOSITION

(71) Applicant: Kramer Laboratories, Inc., Coral Gables, FL (US)

(72) Inventor: Pinaki R. Majhi, Sharon, MA (US)

(73) Assignee: Kramer Laboratories, Inc., Coral Gables, FL (US)

(21) Appl. No.: 15/967,303

(22) Filed: Apr. 30, 2018

Related U.S. Application Data
(60) Provisional application No. 62/492,476, filed on May 1, 2017.

(51) Int. Cl. A61K 31/78; A61P 31/10; A61K 9/00; A61K 45/06

(52) U.S. Cl. CPC A61K 31/78; A61P 31/10; A61K 31/27; A61K 45/06; A61K 2300/00; A61K 9/0014

(57) ABSTRACT

A topical composition for treatment of conditions marked by elevated skin pH may include a partially neutralized carbomer vehicle neutralized to a pH less than or equal to 5. The topical composition may be formulated to undergo continued neutralization in situ to lower skin pH toward healthy levels. The topical composition may be water-based, substantially alcohol free or both. The topical composition may include active medication such as one or more antifungal actives, natural antimicrobial agents such as oils, or a combination thereof.

### CROSS-REFERENCE TO RELATED APPLICATIONS

This patent application claims priority to U.S. Provisional Patent Application No. 62/492,476, filed May 1, 2017, the content of which is hereby incorporated herein by reference.

### TECHNICAL FIELD

The present disclosure relates to topical compositions and methods for the treatment of conditions marked by elevated skin pH such as fungal infections of the skin.

### BACKGROUND

Skin includes a thin protective layer along its surface, which is referred to as the acid mantle. When healthy, the acid mantle has an acidic pH between 4.5 and 6, but more typically around 5.5. The acidic environment is created by sebum excreted from sebaceous glands of the skin, which further mix with lactic and amino acids from sweat.

Conditions such as skin infections may be accompanied by elevated pH along the acid mantle. One such condition is a fungal skin infection caused by the fungi, Trichophyton rubrun (T. rubrun), which is the most predominant and common cause of athlete's foot, fungal nail, jock itch, and ringworm conditions. T. rubrun is a dermatophyte fungus that colonizes in the upper layers of dead skin where it increases the pH of the acid mantle. The alkaline environment contributes to the deterioration of the nail plate. Research shows that the many T. rubrun species create the alkaline environment by release of different amounts of ammonium ion in the skin under different conditions thereby altering the pH to an alkaline range.

### SUMMARY

The present disclosure describes a topical composition and methods of making and using the topical composition to treat a subject in need thereof. The topical composition may be formulated with capacity to undergo in situ neutralization when applied to a skin surface having an elevated skin pH. This in situ neutralization scheme may operate to lower the local pH along the acid mantle as well as maintain a lower pH upon subsequent introduction of alkali or bases.

The topical composition may include partially neutralized carbomer. Upon administration to skin having an elevated skin pH, the partially neutralized carbomer may undergo continued neutralization in situ. For example, when applied to skin having an elevated pH, amine hydroxide ions present in the skin environment may react with the partially neutralized carbomer to further neutralize the carbomer, thereby, lowering the local pH along the acid mantle. The carbomer may have further neutralization capacity to maintain lower pH upon subsequent introduction of alkali or bases.

In various embodiments, the topical composition includes partially neutralized carbomer in a gel, emulsion (w/o, o/w), lotion, cream, emulgel, foam, or other topical format. In some embodiments, the topical composition may be water-based, prepared without alcohols, or both. The topical composition may include oils. In one example, the topical composition is water-based and is prepared with oils to formulate an emulsion. In one such example, the topical composition does not include alcohols. In at least one embodiment, the topical composition is oil based and may be formulated in an ointment dosage form.

In various embodiments, the topical composition may comprise a standalone product without active medication, a topical vehicle carrier for delivering active medication, or may be formulated in a dosage form carrying active medication. In one embodiment, the topical composition is formulated for carrying water soluble or poorly water soluble active medications. For example, the topical composition may comprise a water-based colloidal system forming an emulsion, such as an emulgel, that may be particularly useful in delivering poorly water soluble active medications. Such poorly water soluble active medications may be dissolved, dispersed, or suspended in an oil phase of the composition, for example. In some embodiments, poorly water soluble active medications may be dispersed or suspend within an aqueous phase. In one embodiment, the topical composition is alcohol free. The active medication may include antimicrobial medication such as antifungal actives. The topical composition may include other active medication as well. In one example, the topical composition includes the antifungal active tolnaftate. Alternatively or in addition to active medication, such as antifungal actives, the topical composition may include natural microbial such as antifungal agents such as, but are not limited to, peppermint, tea tree oil, Juglans nigra, aloe, camphor, lavender oil, manuka, or a combination thereof.

The topical composition may be used to treat or prevent conditions that are marked by elevated skin pH. For example, the topical composition may be used in an in situ neutralization scheme as described herein to treat skin conditions marked by pH above 5.5. In a further example, the topical composition may be used in an in situ neutralization scheme to deliver antifungal actives or agents, or both, to treat fungal skin infections marked by a pH above 5.5 or 6.

In one embodiment, the topical composition may be formulated as an emulgel for the treatment of affected skin, e.g., a fungal skin infection such as athlete's foot. Active medication comprising an antifungal active, such as tolnaftate, may be combined with a vehicle composition that includes water, partially neutralized (pH≤5) carbomer, oils having antifungal or skin repair properties, such as tea tree oil, lavender oil, olive oil, eucalyptol, emollients, and emulsifiers. Average skin pH is approximately 5.5; however, fungal infected skin may be exposed to elevated pH. When the topical composition comprising the partially neutralized carbomer vehicle and active medication is applied to the affected skin, the composition is exposed to the basic environment, promoting in situ neutralization of the carbomer. Foot fungi may continue to generate amines after application of the composition, providing a basic and neutralizing environment for the continued in situ neutralization of the carbomer. The effect is that the composition may treat the infection with the antifungal active, agent, or both while also reducing the pH at the affected skin. As the carbomer may possess capacity to undergo continued in situ neutralization, the topical composition may act as a sink for subsequent introduction of alkali or base into the environment to support restoration of healthy pH.

In one aspect, the topical composition includes 0.5% to 4% partially neutralized carbomer that is partially neutralized to a pH less than or equal to 5. In one example, the carbomer may be partially neutralized to a pH less than 5. In this or another example, the topical composition may include 1% to 2% carbomer. In a further aspect, the topical composition may be water-based, substantially free of alcohol, or both. In one example, the topical composition is formulated in a gel, emulsion, or emulgel format. The topical composition may include one or more natural antifungal agent oils or derivatives thereof, active medications such as one or more antifungal actives, or combinations thereof. In one example wherein the topical composition includes one or more antifungal actives, at least one antifungal active is poorly water soluble. In one such example, the poorly water soluble antifungal active comprises tolnaftate. In the above or another example, the topical composition includes one or more natural antifungal agent oils or derivatives selected from peppermint, tea tree, geranium, cassia, Juglans nigra, cinnamon bark, aloe, camphor, lavender, manuka, olive, coconut, lemongrass, clove, oregano, or thyme. In a further aspect, a method of treating a condition marked by elevated skin pH may include administering to affected skin of a subject in need thereof the topical composition described herein. In one example method, the condition may include a topical fungal infection.

### BRIEF DESCRIPTION OF THE DRAWINGS

Various features and characteristics of the non-limiting and non-exhaustive embodiments disclosed and described in this specification may be better understood by reference to the accompanying figures, in which:

FIGS. 1A & 1B present stability testing of a topical composition according to various embodiments described herein.

### DESCRIPTION

Skin health is stabilized at an acidic pH that averages approximately 5.5. Skin with a slightly acidic pH, e.g., below 5 or 5.5, is in better condition to maintain skin health than skin having higher pH, e.g., above a pH of 5 or 5.5, due to beneficial biophysical skin factors supported within the more acidic range such as barrier function, miniaturization, and scaling. However, some conditions are marked by elevated skin pH. The elevated pH accompanying these conditions interferes with healthy skin processes. For instance, when skin becomes infected, such as due to a fungal infection, skin surface pH along the acid mantle may increase. Whereas a pH below 5 (e.g., 4-4.5) promotes resident skin microflora (good bacteria) to become more adhered to the skin, alkaline pH (e.g., 8-9) allows for micoflora dispersion from the skin. Conditions marked by elevated pH may include infections, diseases, or abnormalities that are affected by or impact skin pH. Examples of conditions marked by elevated pH may include, for example, skin conditions such as microbial related skin infections including fungal infections such as, but are not limited to, mycosis (e.g., onychomycosis, dermatophytoses, tinea) and candidiasis. Further examples, include, but are not limited to, irritant contact dermatitis, atopic dermatitis, and acne vulgaris.

The present disclosure describes various embodiments of a topical composition for treatment of conditions marked by elevated pH. The topical composition may include or consists of a partially neutralized vehicle comprising an inactive agent configured to help restore the healthy pH balance of the skin upset by the condition. The inactive agent may include partially neutralized carbomer.

Carbomer is also known as polyacrylic acid (PAA) polymer and is commercially known as Carbopol® polymer. Carbopol® polymer is a registered trademark of The Lubrizol Corporation, and denotes a family of carbomer used as thickeners, suspending agents, and stabilizers. According to various embodiments, the topical composition may include any type of carbomer. For example, the topical composition may include carbomer comprising one or more members of the Carbopol® polymer family including, but not limited to, homopolymers (e.g., acrylic acid cross-linked with allyl sucrose or allyl pentaerythritol) or copolymers (e.g., acrylic acid and C10-C30 alkyl acrylate cross-linked with allyl pentaerythritol).

To build viscosity within a carbomer medium, carbomer, having an un-neutralized pH around 2.5-3.5, may be neutralized with an alkali, such as sodium hydroxide or amine base. The neutralization drives polymer crosslinking thereby increasing viscosity. Depending on the representative proportion of carbomer present in the medium, high viscosities may be achieved within a broad pH range of 5.0-9.0. In various embodiments, carbomer may be present in the topical composition in an amount between 0.5% and 5%, such as between 0.5% and 3% or between 1% and 2% by weight. Greater or lesser amounts may also be used.

The topical composition may be formulated in any suitable topical format. For example, in some embodiments, the topical composition may be formulated as a gel, emulsion (o/w, w/o), lotion, cream, emulgel, or foam.

In various embodiments, the topical composition may include between 0% and 99%, such as 70% to 90%, water by weight. The topical composition, for example, may be formulated in a water-based format comprising greater the 50% water by weight. In some embodiments, the topical composition includes one or more oils. As introduced above, the topical composition may include a dispersion of water and oil forming an emulsion. In one example, the topical composition is formulated as an emulgel. An emulgel is a colloidal system where oil is dispersed in a gel medium forming an emulsion. In at least one embodiment, the topical composition may be formulated as an ointment.

In various embodiments, the topical composition includes partially neutralized carbomer at a pH of ≤5, ≤5, ≤4.9, ≤4.8, ≤4.7, ≤4.6, ≤4.5, ≤4.4, ≤4.3, ≤4.2, ≤4.1, or ≤4.0. In some embodiments, the carbomer may be partially neutralized to a higher or lower pH between 3.5 and 4, between 5 and 5.5 or 6, or between 6 and 6.5 or 7. The pH of topical composition or phase thereof, such as an aqueous phase, may be similar to that of the extent of the neutralization of the carbomer.

As introduced above, the topical composition may be employed in an in situ neutralization scheme designed to lower skin pH when administered to skin having an elevated pH. Upon administration to skin, the partially neutralized carbomer may undergo continued neutralization in situ. For example, when applied to skin having an elevated pH, hydroxide ions present in the skin environment react with the carbomer to neutralize the same, thereby lowering the local pH. The carbomer may have further neutralization capacity to maintain lower pH upon subsequent introduction of alkali or base at the skin surface.

The topical composition may include various excipients. In various embodiments, the topical composition may include excipients such as emulsifiers, stabilizers, or surfactants, which may include Lipo GMS Pure 450 Veg; silicones such as dimethicone; anionic surfactants; nonionic surfactants such as glyceryl stearate, glyceryl laurate, polyethylene glycols, polypropylene glycols, polyethylene glycols, glucosides, poloxamers; or other suitable emulsifiers, stabilizers, or surfactants. Additional components of the topical composition may include, but are not limited to, preservatives or germicides (e.g., phenoxyethanol), buffers, or emollients (e.g., moisturizers, oils, silicone oils). The topical composition may include certain emollients, such as oils, that may also possess antimicrobial properties, as described in more detail below. The topical composition may also include various solvents such non-polar, polar aprotic (e.g., DMSO), polar protic (e.g., water, alcohols) solvents. The topical composition may also include penetrant enhancers such sulfoxides, such as dimethylsulfoxide (DMSO); ethers; surfactants such as sodium laurate, sodium lauryl sulfate, cetyltrimethylammonium bromide, benzalkonium chloride, poloxamer, Tween, and lecithin; alcohols; fatty acids and esters thereof; organic acids; amides; or polyols and esters thereof such as propylene glycol, ethylene glycol, and glycerol.

In various embodiments, the topical composition may be formulated as a standalone product without active medication. In this or another embodiment, the topical composition may be formulated as a topical vehicle carrier for delivering active medication (such as a pharmaceutical compounding base). In another or further embodiment, the topical composition may be formulated in a dosage form carrying active medication.

In various embodiments, the topical composition may be formulated for carrying water soluble or poorly water soluble active medications. For example, the topical composition may comprise a water-based emulgel particularly useful in delivering poorly water soluble active medications. For example, poorly water soluble actives may be dissolved, dispersed, or suspended in an oil phase. In this or another example, all or a portion of the topical composition may be formulated as a gel configured to quickly absorb into the skin. In one such embodiment, the topical composition is alcohol free, which may reduce stinging when applied to irritated or broken skin such as open fissures.

As introduced above, the topical composition may include active medication. The amount of active medication may be between 0 and 99% by weight of the topical composition. The active medication may include, but are not limited to, one or more actives of one or more medications selected from antimicrobial, anti-inflammatory, local anesthetic, or combinations thereof. In one example, the topical composition includes one or more antifungal actives. In a further example, the topical composition includes one or more antifungal actives and at least one additional medication selected from one or more anti-inflammatory actives, local anesthetic actives, or a combination thereof.

In various embodiments, the topical composition includes antimicrobial medication comprising antifungal medication. The antifungal medication may include one or more antifungal actives selected from abafungin, albaconazole, amorolfin, amphotericin b, anidulafungin, bifonazole, butenafine, butoconazole, candicidin, caspofungin, ciclopirox, clotrimazole, econazole, fenticonazole, filipin, fluconazole, flucytosine, griseofulvin, haloprogin, hamycin, isavuconazole, isoconazole, itraconazole, ketoconazole, micafungin, miconazole, naftifine, natamycin, nystatin, omoconazole, oxiconazole, polygodial, posaconazole, ravuconazole, rimocidin, sertaconazole, sulconazole, terbinafine, terconazole, tioconazole, tolnaftate, undecylenic acid, voriconazole, or combination thereof. In one example, the antifungal medication comprises as least on azole. Some embodiments may include other antifungal actives in addition to or instead of the antifungal actives listed above.

In various embodiments, the topical composition includes an antimicrobial medication comprising antibiotic medication. The antibiotic medication may include one or more antibiotic actives selected from amikacin, amoxicillin, gentamicin, kanamycin, neomycin, netilmicin, paromomycin, tobramycin, geldanamycin, herbimycin, carbacephem (loracarbef), ertapenem, doripenem, imipenem, cefadroxil, cefazolin, cefalotin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, clarithromycin, clavulanic acid, clindamycin, colistimethate teicoplanin, azithromycin, dirithromycin, erythromycin, troleandomycin, telithromycin, aztreonam, ampicillin, azlocillin, bacampicillin, carbenicillin, cloxacillin, dicloxacillin, flucloxacillin, mezlocillin, meticillin, nafcillin, norfloxacin, oxacillin, penicillin G, penicillin V, piperacillin, pvampicillin, pivmecillinam, ticarcillin, bacitracin, colistin, colimycin, polymyxin B, ciprofloxacin, enoxacin, gatifloxacin, levofloxacin, lomefloxacin, moxifloxacin, ofloxacin, trovafloxacin, grepafloxacin, sparfloxacin, afenide, prontosil, sulfacetamide, metronidazole, sulfamethizole, sulfanilimide, sulfamethoxazole, sulfisoxazole, trimethoprim, trimethoprim-sulfamethoxazole, demeclocycline, doxycycline, oxytetracycline, tetracycline, arsphenamine, chloramphenicol, chlorhexidine, lincomycin, ethambutol, fosfomycin, furazolidone, isoniazid, linezolid, mupirocin, nitrofurantoin, platensimycin, pyrazinamide, quinupristin/dalfopristin, rifampin, thiamphenicol, rifampicin, minocycline, sultamicillin, sulbactam, sulphonamides, mitomycin, spectinomycin, spiramycin, roxithromycin, meropenem, or combination thereof. Some embodiments may include other antibiotic agents in addition to or instead of the antibiotic actives listed above.

In various embodiments, the topical composition includes anti-inflammatory medication comprising topical steroid medication. The topical steroid medication may include one or more topical steroid actives selected from alclometasone, amcinonide, betamethasone, clobetasol, desonide, desoximetasone, diflorasone, fluocinonide, fluocinolone, flurandrenolide, fluticasone, halcinonide, halobetasol, halometasone, hydrocortisone, mometasone, triamcinolone, or combination thereof. Some embodiments may include other topical steroid actives in addition to or instead of the topical steroid actives listed above.

In various embodiments, the topical composition includes anti-inflammatory medication comprising non-steroidal anti-inflammatory (NSAID) medication. The NSAID medication may include one or more anti-inflammatory actives selected from aceclofenac, acemetacin, acetylsalicylic acid, azathioprine, diclofenac, diflunisal, fenbufen, fenoprofen, flurbiprofen, ibuprofen, indometacin, ketoprofen, leflunomide, lornoxicam, mefenamic acid, meloxicam, naproxen, phenylbutazone, piroxicam, sulphasalazine, tenoxicam, zomepirac, propionic acid such as alminoprofen, apazone, benoxaprofen, butibufen, carprofen, diclofenac, difenpiramide, diflunisal, etodolac, fenbufen, fenoprofen, flurbiprofen, ibuprofen, indomethacin, indoprofen, ketoprofen, ketorolac, meclofenamate, nabumetone, naproxen, oxaprozin, phenylbutazone, piroxicam, pirprofen, pranoprofen, suprofen, sulindac, tolmetin or combination thereof. Some embodiments may include other NSAID actives in addition to or instead of the NSAID actives listed above.

In various embodiments, the composition includes local anesthetic medication comprising one or more local anesthetic actives selected from lidocaine, prilocaine, benzocaine, bupivacaine, tetracaine, articaine, or combination thereof. Some embodiments may include other local anesthetic actives in addition to or instead of the local anesthetic actives listed above.

As introduced above, the topical composition may include one or more oils having beneficial skin properties, antimicrobial properties, or both. For example, the topical composition may include natural antimicrobial agents comprising oils or derivatives thereof. Such natural antimicrobial agents may have antifungal properties, antibiotic properties, or both. The topical composition may include between 0% and 70%, such as 0.01% to 20%, 0.5% to 10%, or 0.5% to 5% natural antimicrobial agent oils or derivatives thereof by weight. In various embodiments, the topical composition includes one or more natural antimicrobial agent oils or derivatives thereof selected from peppermint, tea tree, geranium, cassia, Juglans nigra, cinnamon bark, aloe, camphor, lavender, manuka, olive, coconut, lemongrass, clove, oregano, thyme, or combination thereof. Some embodiments may include other natural antimicrobial agent oils or derivatives thereof in addition to those listed above.

When further combined with clinically proven antifungal actives, as described above and elsewhere herein, the topical composition may include a fusion of natural healing ingredients with antimicrobial medication. For example, when further combined with clinically proven antifungal actives, such as tolnaftate, the topical composition may be formulated with a variety of natural fungus inhibiting ingredients to provide a potent fungus killing composition. In one such example, the topical composition includes one or more natural antifungal oils or derivatives thereof selected from peppermint, tea tree, geranium, cassia, Juglans nigra, cinnamon bark, aloe, camphor, lavender, manuka, olive, coconut, lemongrass, clove, oregano, thyme, or combination thereof.

The topical composition may be formulated to include any number of active over the counter medications, natural agents, or combinations thereof to assist restoration of the skin's natural, healthy pH level.

Methods of Making a Topical Composition

The present disclosure also includes methods of making the topical composition. The topical composition comprising partially neutralized carbomer for in situ neutralization may be formulated in any suitable topical format, such as a gel, emulsion (w/o, o/w), lotion, cream, emulgel, foam, ointment, or other topical format. In some embodiments, the topical composition comprising partially neutralized carbomer for in situ neutralization may be formulated in a topical format selected from a lotion, cream, solution, spray, or foam. In one embodiment, the topical composition coats, layers, infuses, or embeds a fabric or other application material for positioning over skin, such as a bandage, wrap, or adhesive bandage.

In various embodiments, the method of making the topical composition may include adding carbomer to a solution, suspension, emulsion, or component or phase thereof. The method may further include partially neutralizing the carbomer to a desired partially neutralized pH with a suitable base. In some embodiments, partially neutralizing the carbomer may occur, at least in part, via addition of the carbomer to the solution, suspension, emulsion, or component or phase thereof. Partially neutralizing the carbomer may also include addition of alkali or base to the solution, suspension, emulsion, or component or phase thereof.

The method may also include addition of one or more other topical composition ingredients such as dispersants, surfactants, preservatives, or emollients before or after addition of the carbomer. The additional ingredients may have various degrees of solubility and may include oil soluble ingredients, water soluble ingredients, or both.

The topical composition may be formulated as a topical vehicle, which may be a standalone treatment or a topical base for pharmaceutical compounding. For example, the partially neutralized carbomer vehicle may be formulated as a topical base for compounding with active medication, natural agents, or both or may be formulated as a topical base for further pharmaceutical compounding that includes active medication, natural agents, or both.

The topical composition may also be formulated to include active medication, one or more natural antimicrobial agents, such as oils having antimicrobial properties, or both, as introduced above, in a stable pharmaceutical product. For example, the method of making the topical composition may include adding active medication before or after adding the carbomer to the solution, suspension, emulsion, or component or phase thereof. The active medication may be suspended, dispersed, dissolved, encapsulated, or combination thereof in the topical composition. In one example, active medications may be dissolved within an oil phase, aqueous phase, or both. Adding the active medication may include dissolving, suspending, or dispersing all or a portion of the active medication in an oil or aqueous phase of the composition. In one example, the topical composition includes the active medication suspended homogenously in the partially neutralized carbomer based medium, such as an emulgel vehicle medium, which may further be water-based. The suspension may also contain other beneficial ingredients, such as ingredients having various levels of solubility with respect to water and oil phases, as described above.

The topical composition may include or be formulated to include any number of active over the counter medications, natural agents, or combinations thereof to assist restoration of the skin's natural, healthy pH level.

In any of the above or another example, the method may include adding oils, such as natural antimicrobial agent oils, before or after adding the carbomer. In various embodiments, when the topical composition includes an oil component, active medication may be added before or after addition of all or a portion of the oil component. When active medication includes poorly water soluble actives, for example, it may be preferable to add such actives after addition of all or part of an oil phase or add such poorly water soluble actives directly to all or a portion of an oil component making up all or a portion of the oil phase before adding the oil component to the aqueous component or phase to form an emulsion. Poorly water soluble actives may also be suspended in all or a portion of a water component making up all or a portion of an aqueous component or phase before or after adding the water component to an oil component or phase to form an emulsion. Surfactants, dispersants, or emulsifiers may also be used to assist in dissolving, suspending, dispersing, or encapsulating the actives. In some embodiments, it may be beneficial to apply heat to components or phases to assist in dissolution, suspension, or dispersion of ingredients in the composition, component, or phase thereof.

A method of pharmaceutical compounding using the topical composition comprising partially neutralized carbomer as a pharmaceutical base vehicle may include suspending, dispersing, or dissolving active medication in the topical composition, e.g., within an oil phase, aqueous phase, or both. All or a portion of the active medication may be encapsulated, wetted, dissolved, or suspended in water, oil, lipids, DMSO, dimethylformamide, dimethylacetamide, acetone, hexane, mineral oil, isopropanol, methanol, or other suitable liquid prior to addition to the topical composition. In one example, active medication may be suspended homogenously in the partially neutralized carbomer medium, such as an emulgel vehicle medium. The suspension may also contain other beneficial ingredients (both oil and water soluble ingredients, for example) as described above. In any of the above or a further example, natural antimicrobial agents such as oils or derivatives thereof may be added instead of or in addition to active medication. The topical composition may include or be compounded to include any number of active over the counter medications, natural agents, or combinations thereof to assist restoration of the skin's natural, healthy pH level. In some embodiments, the topical composition is formulated in a topical format selected from a lotion, cream, solution, spray, or foam. In one embodiment, a fabric or other application material for positioning over skin, such as a bandage, wrap, or adhesive bandage is coated, layered, infused, or embedded with or by the topical composition.

Table 1 illustrates ingredients of a topical composition formulated as a fungi nail natural fusion antifungal gel according to various embodiments described herein. A method of making the composition of Table 1 may include combining poloxamer, dimethicone, phenoxetol, Lipo GMS Pure 450 Veg, carbopol, and water in amounts shown in Table 1. The method may also include combining natural antimicrobial agents tea tree oil, aloe vera gel, lavender oil, eucalyptol, and olive oil in amounts shown in Table 1. Tolnaftate may be combined in an amount of 1% by wt; however, other amounts may be used, e.g., between 0.01% and 5% by weight. Tolnaftate may be provided in oil or powder form. The tolnaftate may be suspended homogenously in the partially neutralized carbomer medium. Tolnaftate is practically insoluble in water making it a challenge to incorporated into a water-based and alcohol-free gel product. The formula contains an oil component comprising a variety of oils that have benefits with respect to treating fungal infected skin or restoring damaged skin. The tolnaftate may be dissolved, suspend, or dispersed in all or a portion of the oil component before or after the oil component or portion thereof is combined with the water. The tolnaftate and oil may be suspended homogenously in the partially neutralized carbomer medium to provide a colloidal system forming an emulsion. The tolnaftate may be suspend in all or a portion of the water before or after formation of the emulsion. For example, tolnaftate suspended in a portion of the water may be added to an emulsion. The pH of the suspension may be adjusted to the desired partially neutralized pH, as described above, such to a pH≤5 with suitable pH adjusting agents. In some embodiments, pH may not require adjustment.

According to one embodiment, a method of making the topical composition of Table 1 includes suspending tolnaftate in a portion of the water with poloxamer to formulate an aqueous drug phase. A homogenous liquid oil phase is also prepared by combining tea tree oil, lavender oil, Eucalyptol, olive oil, dimethicone, and Lipo GMS pure 450 with application of heat (final temperature 60-65° C.) and mechanical agitation. Carbopol is combined with a second portion of the water containing aloe vera to form an aqueous carbomer phase, which is heated to 60-65° C. The aqueous carbomer phase and the homogenous liquid oil phase are then mixed with agitation to form an emulsion. The emulsion is cooled to 45-45° C. and the aqueous drug phase is added and mixed to homogenously distribute the drug particles within the emulsion. The emulsion is then cooled to 25-30° C. and phenoxyethanol (preservative) is added and mixed thoroughly. Sodium hydroxide solution is then added to partially neutralize the carbomer to a targeted final pH of 5 or below. In this example, the partially neutralized pH is approximately 4.4. Coloring agents may also be added to provide a desired appearance. For example, the topical composition according to Table 1 includes green and yellow coloring agents to provide a light green thin gel.

TABLE 1

| INGREDIENT | Wt. % (range) |
|---|---|
| Tolnaftate (active) | 1.00 |
| Poloxamer 188 | 0.05-0.2 |
| Purified Water USP | 90-98 |
| Tea Tree Oil | 0.01-1.0 |
| Aloe Vera Gel | 0.01-1.0 |
| Lavender Oil | 0.01-1.0 |
| Eucalyptol | 0.01-1.0 |
| Olive Oil | 0.01-1.0 |
| Dimethicone 350 NF | 0.02-1.0 |
| Carbopol 981 | 1-2 |
| Phenoxetol | 0.3-1.0 |
| Lipo GMS Pure 450 Veg | 0.05-2.0 |
| D&C Green #5 | 0-0.0005 |
| D&C Yellow #10 | 0-0.0005 |

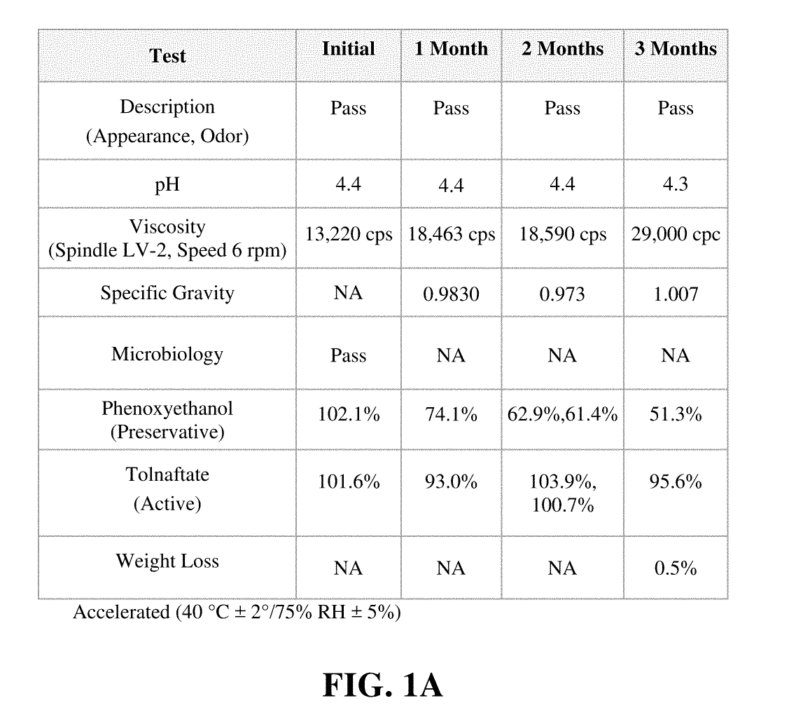

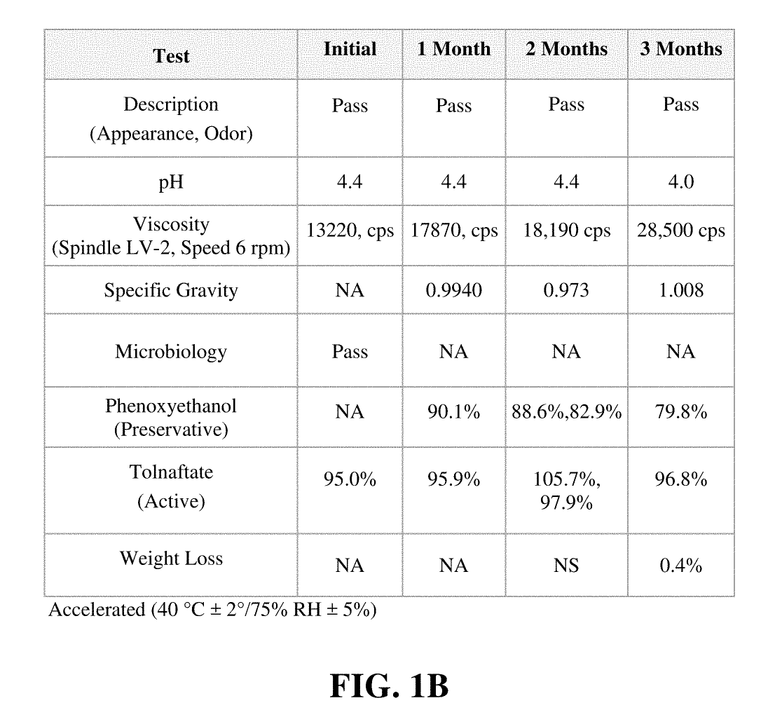

As introduced above, the topical composition may be formulated to provide a stable composition. FIGS. 1A & 1B present results of stability tests performed with respect to a topical composition according to Table 1. As can be seen, the composition maintains appearance, scent, pH, structure, and potency of the active ingredient over the test periods.

Methods of Treatment

The topical composition may be administered to a subject in need thereof to treat a condition marked by elevated skin pH. The subject may typically be an organism such as an animal or human subject. Conditions marked by elevated pH may include infections, diseases, or abnormalities that are affected by or impact, e.g., accompany or cause, elevated skin pH. Examples of conditions marked by elevated pH may include, for example, skin conditions such as microbial related skin infections including fungal infections, which may include, but are not limited to, mycosis (e.g., onychomycosis, dermatophytoses, tinea) and candidiasis. Further examples, may include, but are not limited to, irritant contact dermatitis, atopic dermatitis, and acne vulgaris.

According to various embodiments, the topical composition may be administered to a subject in need thereof to treat a condition marked by elevated skin pH, e.g., conditions that raise the pH level of skin beyond its healthy stated, as noted above. The topical composition may be topically applied for restoration of healthy skin pH levels. Example conditions may include treatment of acne or bacterial infections as introduced above. Other example conditions include psoriasis, dandruff, dry skin or eczema. In one embodiment, the topical composition may be utilized to treat scalp itch, which may be scalp itch caused by dry scalp or dandruff. Further example conditions include skin inflammation conditions. Further example conditions include bug bites, sun burns, and allergy related conditions.

A method of treating a condition marked by elevated skin pH may include administering the topical composition described herein to a subject in need thereof. For example, the topical composition may include partially neutralized carbomer and, optionally, active medication, natural antimicrobial agents, or both. The topical composition may include or be formulated to include any number of active over the counter medications, natural agents, or combinations thereof to assist restoration of the skin's natural, healthy pH level. The topical composition may be applied to the skin area to be treated such as a foot, arm, trunk, face, vaginal region, groin, posterior, or other skin area.

According to various embodiments, the topical composition may be administered daily as required to treat the condition or as otherwise directed. The topical composition may be administered one or more times a day or as otherwise directed. The amount of topical composition administered may depend on the surface area of the skin onto which it is to be applied. As noted above, the topical composition may be formulated to quickly absorb onto the skin. In some embodiments, the topical composition may be formulated to coat the skin surface. In one such example, an occlusive patch may be placed over the application area.

In one embodiment, a method of treating a toe/nail fungal infection includes administering the topical composition described herein to a subject in need thereof. The topical composition may be applied to an affected skin area, which may include an affected nail area. For example, the topical composition may include a balanced ingredient composition that is formulated to provide optimal pH conditions along the acid mantle to encourage skin health mechanisms while also treating the fungal infection. The topical composition may include partially neutralized carbomer and active medication comprising one or more antifungal actives, one or more natural antifungal agents, or a combination thereof. For example, the antifungal active may include tolnaftate and the natural antifungal agent may include one or more natural antifungal oils or derivatives thereof selected from peppermint, tea tree, geranium, cassia, Juglans nigra, cinnamon bark, aloe, camphor, lavender, manuka, olive, coconut, lemongrass, clove, oregano, thyme, or combination thereof. In one embodiment, the method of treating a toe/nail fungal infection may include applying the topical composition of Table 1 to an affected skin region, which may include an affected nail region.

Before treatment, the damaged skin may have an elevated pH compared to its normal pH range (e.g., 5 to 6). Upon application to the damaged skin, neutralization of the partially neutralized carbomer may continue. After the topical composition is applied to the damaged skin, the fungi (e.g., T. rubrun) may continue to generate amines as it continues to grow, thus, providing a basic and neutralizing environment for the carbomer. As the in situ neutralization of the carbomer continues on the acid mantle, pH of the affected skin may be reduced. After completion of the treatment period, the skin may be returned to its healthy pH range and the fungal infection cured.

Various embodiments are described and illustrated in this specification to provide an overall understanding of the composition, function, operation, and application of the disclosed compositions and methods. It is understood that the various embodiments described and illustrated in this specification are non-limiting and non-exhaustive. Thus, the invention is not necessarily limited by the description of the various non-limiting and non-exhaustive embodiments disclosed in this specification. The features and characteristics illustrated or described in connection with various embodiments may be combined with the features and characteristics of other embodiments. Such modifications and variations are intended to be included within the scope of this specification. As such, the claims may be amended to recite any features or characteristics expressly or inherently described in, or otherwise expressly or inherently supported by, this specification. Further, Applicant reserves the right to amend the claims to affirmatively disclaim features or characteristics that may be present in the prior art. Therefore, any such amendments comply with the requirements of 35 U.S.C. §§ 112(a) and 132(a). The various embodiments disclosed and described in this specification can comprise, consist of, or consist essentially of the features and characteristics as variously described in this specification.

Also, any numerical range recited in this specification is intended to include all sub-ranges of the same numerical precision subsumed within the recited range. For example, a range of “1.0 to 10.0” or between “1.0 and 10.0” is intended to include all sub-ranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited in this specification is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited in this specification. All such ranges are intended to be inherently described in this specification such that amending to expressly recite any such sub-ranges would comply with the requirements of 35 U.S.C. §§ 112(a) and 132(a). Any numerical descriptions in this specification are intended to include minor deviations including reasonable equivalents.

The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an implementation of the described embodiments. Further, the use of a singular noun includes the plural, and the use of a plural noun includes the singular, unless the context of the usage requires otherwise. The grammatical conjunction “or” is generally used in this specification as inclusive, generally synonymous with “and/or”. By way of example, “x” or “y” may mean “x”, “y”, both “x” and “y”.

What is claimed is:

1. A topical composition comprising: 0.5% to 4% carbomer by weight, wherein the carbomer is partially neutralized to a pH less than or equal to 5.

2. The topical composition of claim 1, wherein the topical composition is water-based.

3. The topical composition of claim 1, wherein the topical composition comprises a gel, emulsion, or emulgel.

4. The topical composition of claim 1, wherein the topical composition is substantially free of alcohol.

5. The topical composition of claim 1, further comprising one or more natural antifungal agent oils or derivatives thereof.

6. The topical composition of claim 1, wherein the topical composition comprises 1% to 2% carbomer by weight.

7. The topical composition of claim 1, wherein the carbomer is partially neutralized to a pH less than 5.

8. The topical composition of claim 1, further comprising active medication, wherein the active medication comprises one or more antifungal actives.

9. The topical composition of claim 8, wherein at least one antifungal active is poorly water soluble.

10. The topical composition of claim 9, wherein the topical composition comprises a water-based gel, emulsion, or emulgel and is substantially free of alcohol.

11. The topical composition of claim 10, further comprising one or more natural antifungal agent oils or derivatives thereof.

12. The topical composition of claim 11, wherein the one or more antifungal actives comprise tolnaftate and the one or more natural antifungal agent oils or derivatives thereof comprise at least one natural antifungal agent oil or derivative thereof selected from peppermint, tea tree, geranium, cassia, Juglans nigra, cinnamon bark, aloe vera, camphor, lavender, manuka, olive, coconut, lemongrass, clove, oregano, or thyme.

13. A method of treating a condition marked by elevated skin pH, the method comprising:
administering to affected skin of a subject in need thereof a topical composition, wherein the topical composition comprises 0.5% to 4% carbomer by weight, and wherein the carbomer is partially neutralized to a pH less than or equal to 5.

14. The method of claim 13, wherein the topical composition is water-based.

15. The method of claim 13, wherein the topical composition comprises a gel, emulsion, or emulgel.

16. The method of claim 13, wherein the topical composition is substantially free of alcohol.

17. The method of claim 13, wherein the condition comprises a fungal skin infection, and wherein the topical composition further comprises one or more natural antifungal agent oils or derivatives thereof.

18. The method of claim 13, wherein the topical composition comprises 1% to 2% carbomer by weight.

19. The method of claim 13, wherein the carbomer is partially neutralized to a pH less than 5.

20. The method of claim 13, wherein the topical composition further comprising active medication, wherein the active medication comprises one or more antifungal actives, and wherein the condition comprises a fungal skin infection.

21. The method of claim 20, wherein at least one antifungal active is poorly water soluble.

22. The method of claim 21, wherein the topical composition comprises a water-based gel, emulsion, or emulgel and is substantially free of alcohol.

23. The method of claim 22, wherein the topical composition further comprises one or more natural antifungal agent oils or derivatives thereof.

24. The method of claim 23, wherein the one or more antifungal actives comprise tolnaftate and the one or more natural antifungal agent oils or derivatives thereof comprise at least one natural antifungal agent oil or derivative thereof selected from peppermint, tea tree, geranium, cassia, Juglans nigra, cinnamon bark, aloe, camphor, lavender, manuka, olive, coconut, lemongrass, clove, oregano, or thyme.
